(19) United States
(12) Patent Application Publication
(10) Pub. No.: US 2021/0275108 A1
(43) Pub. Date: Sep. 9, 2021

(54) METHOD AND SYSTEM FOR GENERATING SYNTHETIC COMPOSITE WAVEFORMS FOR DETERMINING INDIVIDUAL PHYSIOLOGICAL STATE

(71) Applicant: ELSRA MedTech Co., Ltd, Taipei (TW)

(72) Inventor: Jing-Sheng YEH, Taipei (TW)

(21) Appl. No.: 16/846,651

(22) Filed: Apr. 13, 2020

(30) Foreign Application Priority Data
Mar. 6, 2020 (TW) 109107544

(51) Int. Cl. A61B 5/00; A61B 5/024; G16H 50/30

(52) U.S. Cl. CPC A61B 5/7278; A61B 5/4809; A61B 5/0456; G16H 50/30; A61B 5/02416; A61B 5/02405

(57) ABSTRACT

The invention generates synthetic composite waveforms for determining an individual's physiological state, and includes steps of: obtaining a periodic physiological signal of an individual within a time frame; analyzing the periodic physiological signal to obtain and to convert peak-to-peak time intervals of the periodic physiological signal into frequencies; establishing a physiological variation waveform according to the variation of the frequencies; defining at least one synthetic basic waveform according to the frequency trend or fluctuation amplitude of the physiological variation waveform; combining at least two synthetic basic waveforms to generate at least one synthetic composite waveform; and defining respectively each of the at least one synthetic composite waveform as a physiological state. The present invention needn't collect many subjects' physiological signals, thereby reducing the amount of information to be analyzed and overcoming the disability to establish a standardized model by statistical analysis methods due to differences among individuals.

### CROSS REFERENCE TO RELATED APPLICATION(S)

This non-provisional application claims the benefit under 35 U.S.C. § 119(e) to patent application No. 109107544 filed in Taiwan on Mar. 6, 2020, which is hereby incorporated in its entirety by reference into the present application.

### BACKGROUND OF THE INVENTION

### 1. Field of the Invention

The present invention relates to a method and a system for generating synthetic composite waveforms for determining an individual's physiological state.

### 2. Description of the Related Art

Sleep disorder refers to abnormalities in falling asleep and/or maintaining normal sleep, or behavioral abnormalities or physiological events that occur during sleep or during sleep arousal transition, which include variations in the amount of sleep manifested as too little sleep or too much sleep. There are also variations in sleep quality, such as sleep Apnea syndrome and sleepwalking. The clinical manifestations of sleep disorders are diverse, with different etiology and pathology respectively. Sleep disorder itself can induce or cause a variety of diseases in the body, and diseases in various systems of the body can be concurrent with or accompanied by sleep disorders. Among them, sleep-related breathing disorder is a disease characterized by abnormal breathing rhythm and/or abnormal airway ventilation during sleep, with or without daytime breathing disorder. Sleep-related breathing disorders include obstructive sleep Apnea, central sleep Apnea, sleep-related hypopnea diseases, sleep-related hypoxemia, snoring, and sleep moaning.

Obstructive Sleep Apnea (OSA) means that the Apnea and the hypopnea are caused by repeated collapse and obstruction of the upper airway during sleep. OSA in turn leads to frequent hypoxemia, hypercapnia, significant fluctuations in intrathoracic pressure, and disturbance of sleep structure, and can lead to long-term impairments in multiple-organ system functions. Patients' snoring and recurrent Apnea during sleep are usually accompanied by symptoms of daytime sleepiness, lack of concentration, emotional disturbance, insomnia, fatigue, etc. Obstructive sleep Apnea is a chronic disease that requires comprehensive multidisciplinary treatments, nevertheless an individualized treatment should be followed. The main causes and risk factors of OSA include: obesity (body mass index BMI≥28 kg/m2), age (incidence rate increases with adult age), gender (more males than females), abnormal upper airway anatomy which includes increased nasal resistance: nasal polyps, nasal cavity tumors, nasal valve stenosis, nasal septum deflection, chronic hypertrophic rhinitis, allergic rhinitis, etc., and hypertrophy of soft tissues of the pharynx: hypertrophy of the tongue, hypertrophy of the tonsils and adenoids, hypertrophy and/or lowering of the soft palate, etc., long-term heavy drinking and/or sedative hypnotics intake, long-term heavy smoking, genetic predisposition, and secondary diseases such as endocrine system diseases, chronic heart dysfunction, craniofacial developmental deformity, etc. Obstructive Apnea is often accompanied by hypopnea, or coexists with central sleep Apnea, and any restriction of airflow caused by airway obstruction or central nervous regulation often causes hypoxemia.

At present, objective methods of sleep diagnosis and evaluation are mainly performed by polysomnography (PSG). Subjects need to sleep overnight in the examination room of the medical institution, and the tests are assisted by a technician. The process includes multiple physiological signal acquisitions, including brain waves, electrooculogram, electromyograms, electrocardiograms, thoracoabdominal respiratory mobility, oral and nasal airflow, snoring, blood oxygen saturation, limb mobility, body posture and synchronized recorded images, etc. These signals are collected by the body surface sensors and recorded by the computer, and finally the results of the examination are interpreted by a sleep medicine specialist. The acquisition of multiple physiological signals of PSG, especially the monitoring of a large number of signals on the skull and face, requires the subject to be affixed with many electrodes, so a large number of wires are disposed around the subject's head, affecting the subject to fall asleep and causing poor subject experiences. In addition, during the test in the examination room, changes in the sleeping environment cause many subjects to change their sleeping behaviors, resulting in differences between the examination results and the sleeping behaviors of the subjects in their natural states. Furthermore, the amount of sleep physiological examinations and the physiological signal analysis information thereof is large and inconvenient for utilization; in addition, the detection system of multiple physiological examinations for sleep is expensive, and it is difficult to promote and use the system. Although the existing Cardiopulmonary Coupling (CPC) technology can reduce the difficulty of collecting multiple physiological signals, the amount of information it analyzes is large and there are certain limitations in the refinement of time intervals. Therefore, there is an urgent need to develop a method that is easy to measure, implement, and truly reflect the physiological state of the subject's natural state.

### SUMMARY OF THE INVENTION

The present invention relates to a synthetic composite waveform for determining an individual's physiological state, and a generating method and a generating system thereof. The present invention collects an individual ECG signal through a lightweight ECG signal detection instrument, analyzes and generates synthetic basic waveforms of the individual's physiological signals and the synthetic basic waveforms combined to generate synthetic composite waveforms, and through the synthetic composite waveforms, analyzes an individual's physiological signals. The method and system of the present invention can be used to detect the physiological state of an individual, including the sleep states of the individual during sleep, the sleep Apnea index, the type of sleep Apnea, the duration of sleep Apnea, the sleep state of the individual when sleep Apnea occurs, and the individual's stable and unstable physiological states during the non-sleep time interval.

The method and system of the present invention analyze individual physiological signals and generate individualized synthetic basic waveforms and synthetic composite waveforms, and then analyze the physiological signals of the individual through the synthetic basic waveforms and the synthetic composite waveforms without collecting physiological signals and statistical analysis of a large number of subjects. In addition to reducing the amount of information to be analyzed, it can also overcome the problem of not being able to establish a standardized model by statistical analysis methods due to differences among individuals.

The implementation location of the method and system for generating synthetic composite waveforms of the present invention is not limited to the examination room of a medical institution, and can be performed in the subject's residential environment as well. In addition, the lightweight instrument also improves the subject's comfort during examination, therefore an individual's physiological status can be truly reflected.

The method and system of the present invention reduce the amount of information to be analyzed after the examination through automated analysis, thereby providing examination results more quickly, and providing references to physicians for the prescription of medication and the like.

A purpose of the present invention is to provide a method for generating synthetic composite waveforms for determining an individual's physiological state, which includes the steps of: (A) obtaining an individual's periodic physiological signal within a time frame; (B) analyzing the periodic physiological signal to obtain peak-to-peak intervals of the periodic physiological signal and converting the peak-to-peak interval into frequencies; (C) establishing a physiological variation waveform according to variations of the frequencies; (D) according to frequency trend or fluctuation amplitude of the physiological variation waveform, defining at least one synthetic basic waveform; (E) combining at least two said synthetic basic waveforms to generate at least one synthetic composite waveform; and (F) respectively defining each one of the at least one synthetic composite waveform as a physiological state.

In one embodiment, the physiological signal is a single signal.

In one embodiment, the physiological signal is an electrocardiogram (ECG) signal or a photoplethysmogram (PPG) signal.

In one embodiment, the fluctuation amplitude refers to a difference between two adjacent sets of signal values.

In one embodiment, the signal values refer to frequencies.

In one embodiment, the synthetic basic waveforms include a stable wave, a state wave, and an irregular wave, wherein the stable wave means that the fluctuation amplitude appears stable, and the state wave means that the fluctuation amplitude gradually rises or gradually falls, and the irregular wave means that the fluctuation amplitude exhibits irregular variations.

In one embodiment, the stable wave is classified into a stable wavelet, a stable medium wave, and a stable big wave according to the fluctuation amplitude.

In one embodiment, the physiological state is a sleeping state or a non-sleeping state.

In one embodiment, the sleeping state and the non-sleeping state are determined by combining an individual's physiological signal waveform and body position information.

In one embodiment, the method for generating synthetic composite waveforms further includes the step of: (G) determining a physiological state of the individual during the time frame.

The present invention also provides a synthetic composite waveform generation system for determining an individual's physiological state, which includes: A. a detection module for obtaining a periodic physiological signal of an individual within a time frame; B. a storage module for storing the physiological signal; C. a computing module for:

c1. analyzing the periodic physiological signal to obtain peak-to-peak intervals of the periodic physiological signal, and converting the peak-to-peak intervals into frequencies;

c2. establishing a physiological variation waveform according to variations in the frequencies;

c3. defining at least one synthetic basic waveform according to frequency trend or fluctuation amplitude of the physiological variation waveform;

c4. combining at least two of the synthetic basic waveforms to generate at least one synthetic composite waveform; and

c5. defining each of the at least one synthetic composite waveform respectively as a physiological state.

In one embodiment, the physiological signal is a single signal.

In one embodiment, the physiological signal is an electrocardiogram (ECG) signal or a photoplethysmogram (PPG) signal.

In one embodiment, the fluctuation amplitude refers to a difference between two adjacent sets of signal values.

In one embodiment, the signal values refer to frequencies.

In one embodiment, the synthetic basic waveform includes a stable wave, a state wave, and an irregular wave, wherein the stable wave means that the fluctuation amplitude appears stable, the state wave means that the fluctuation amplitude gradually rises or gradually falls, and the irregular wave means that the fluctuation amplitude exhibits irregular variations.

In one embodiment, the stable wave is classified into a stable wavelet, a stable medium wave, and a stable big wave according to the fluctuation amplitude.

In one embodiment, the physiological state is a sleeping state or a non-sleeping state.

In one embodiment, the sleeping state and the non-sleeping state are determined by combining an individual's physiological signal waveform and body position information.

In one embodiment, the system for generating a combined composite waveform further includes an output module for outputting a physiological state of the individual during the time frame.

### BRIEF DESCRIPTION OF THE DRAWINGS

FIG. 1A is a flowchart of generating a synthetic basic waveform and a synthetic composite waveform by using an ECG signal according to an embodiment of the present invention;

FIG. 1B is a composition diagram of a typical ECG signal;

FIG. 2A is a flowchart of generating a synthetic basic waveform and a synthetic composite waveform by using a photoplethysmography (PPG) signal according to an embodiment of the present invention;

FIG. 2B is a composition diagram of a typical photoplethysmography (PPG) signal;

FIG. 3 is an example of a synthetic basic waveform according to an embodiment of the present invention;

FIG. 4 is an example of a synthetic composite waveform according to an embodiment of the present invention;

FIG. 5 is a physiological state analysis system according to an embodiment of the present invention.

### DETAILED DESCRIPTION OF THE INVENTION

In the following, the technical solutions in the embodiments of the present invention will be clearly and fully described with reference to the drawings in the embodiments of the present invention. Obviously, the described embodiments are only a part of the embodiments of the present invention, but not all the embodiments. Based on the embodiments of the present invention, all other embodiments obtained by a person of ordinary skill in the art without creative efforts shall fall within the protection scope of the present invention.

The “individuals” described herein include, but are not limited to, human individuals; SAHS refers to Sleep Apnea Hypopnea Syndrome; AHI refers to sleep Apnea-Hypopnea index i.e. number of Apnea plus Hypopnea per hour during sleep.

### Embodiment 1: Generation of Synthetic Basic Waveforms and Synthetic Composite Waveforms

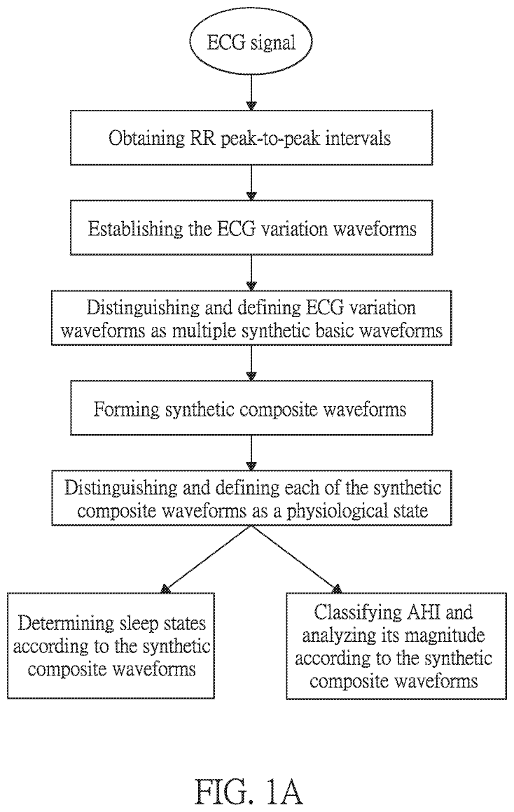

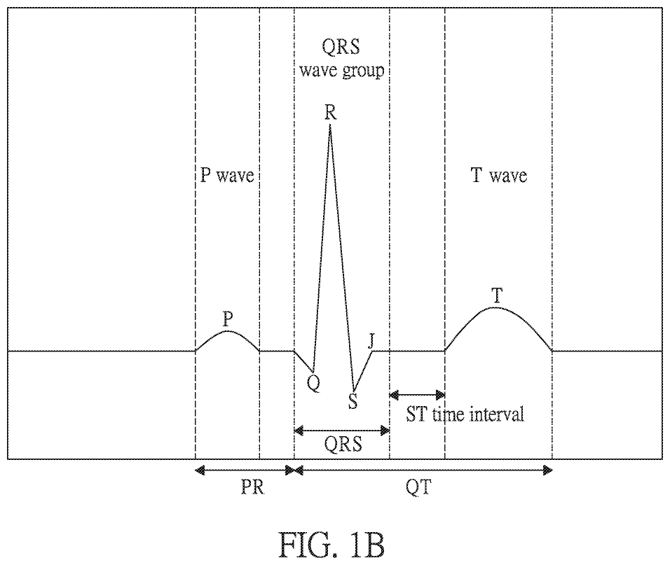

The synthetic basic waveforms and the synthetic composite waveforms of the present invention can be generated through different periodic physiological signals. FIG. 1A is a flowchart of generating synthetic basic waveforms and synthetic composite waveforms by using an ECG signal according to an embodiment of the present invention. Obtain the ECG signal of the individual within a time frame, analyze the ECG signal to obtain the RR peak-to-peak intervals (FIG. 1B), convert them into frequencies, and then plot the frequency variations per second to obtain the ECG variation waveform. Next, the ECG variation waveforms are distinguished according to its frequency trend or fluctuation amplitude, and are defined as multiple synthetic basic waveforms. Combine at least two synthetic basic waveforms to form at least one synthetic composite waveform and respectively define each of the at least one synthetic composite waveform as a physiological state. Finally, use the generated synthetic basic waveforms and synthetic composite waveforms to determine the physiological state of the individual.

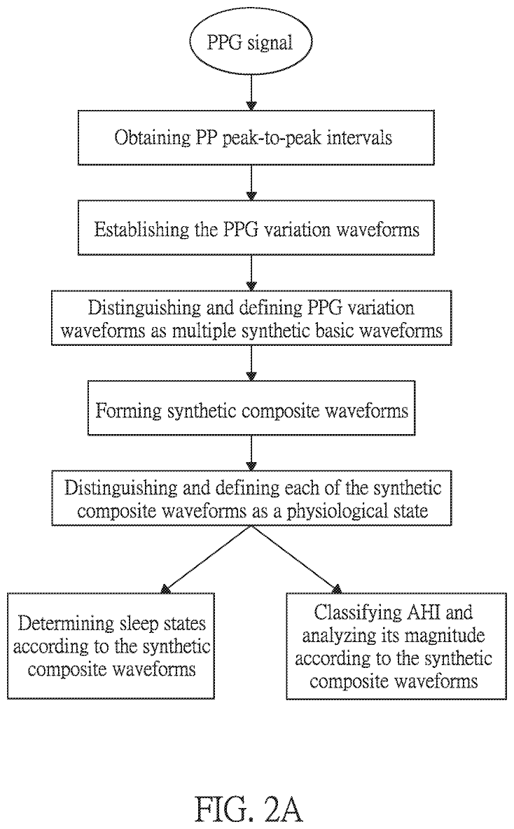

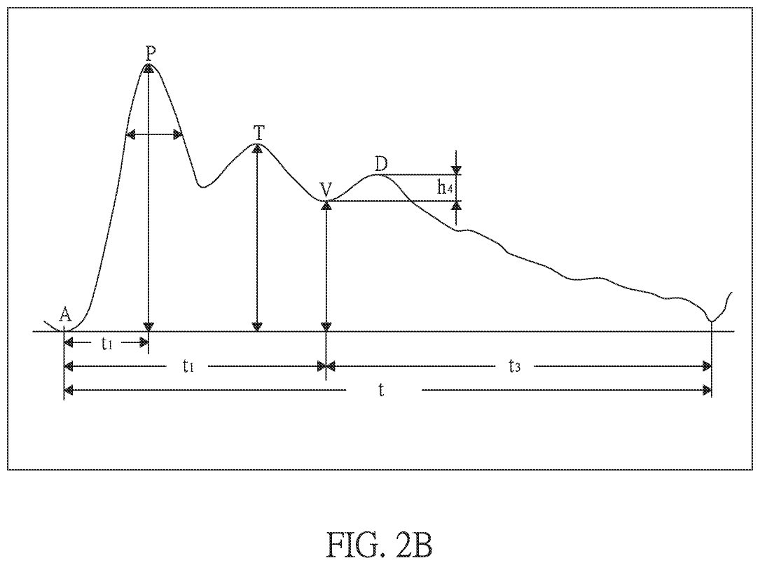

FIG. 2A is a flowchart of generating a synthetic basic waveform and a synthetic composite waveform by using a photoplethysmography (PPG) signal according to an embodiment of the present invention. Obtain the PPG signal of the individual during a time frame, analyze the PPG signal to obtain the PP peak-to-peak intervals (FIG. 2B), and convert them into frequencies, and then plot the frequency variations per second to obtain the PP interval variation waveform. Next, the PP interval variation waveform is distinguished according to its frequency trend or fluctuation amplitude, and is defined as a variety of synthetic basic waveforms. Combine at least two synthetic basic waveforms into at least one synthetic composite waveform and respectively define each of the synthetic composite waveform as a physiological state. Finally, use the generated synthetic basic waveforms and synthetic composite waveforms to determine the physiological state of the individual.

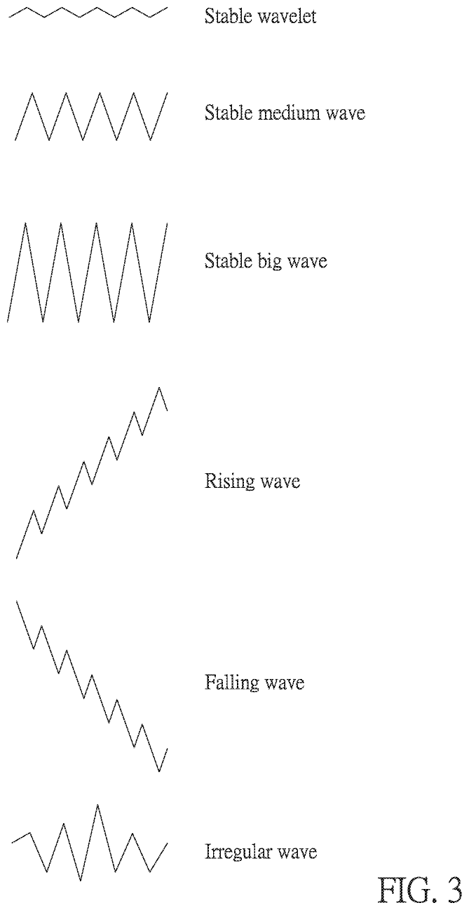

Analysis of the difference between two adjacent sets of signal values (hereinafter referred to as fluctuation amplitude) or fluctuation trend for the ECG variation waveform or the PPG variation waveform can obtain several synthetic basic waveforms as shown in FIG. 3, including stable waves, state waves, and irregular waves. Among them, the stable waves are divided into stable wavelet, stable medium wave and stable big wave, and the state waves are divided into rising wave and falling wave. In this embodiment, stable wavelets are defined as those with fluctuation amplitudes less than or equal to 3, stable medium waves are those with fluctuation amplitudes greater than 3 and less than or equal to 7, and stable big waves are those with fluctuation amplitudes greater than 7; rising waves are those whose fluctuation trends gradually rise, and falling waves are those whose fluctuation trends gradually fall, and an irregular wave means that the difference in the value of two or two sets of adjacent signals exhibits irregular variations.

Since there is a coupling relationship between the individual's ECG and respiration and such relationship will also show different coupling characteristics in different states, each synthetic basic waveform corresponds to one respective state.

Taking the sleep states as an example, the correspondences between each sleep state and each synthetic basic waveform are shown in Table 1.

TABLE 1
Correspondences between synthetic basic waveforms
and sleep states and identification features

| Composition of waves | Sleep state | Identification feature |
|---|---|---|
| Stable wavelet | Stable (deep sleep, | Fluctuation amplitude ≤ 3 |
| | light sleep, activity) | |
| Stable medium wave | Light sleep, dream | 7 ≥ fluctuation amplitude > 3 |
| Stable big wave | Waking, SAHS | Fluctuation amplitude > 7 |
| Irregular wave | Unstable (light sleep, | Nonuniform fluctuation |
| | waking, activity) | amplitudes |
| Rising wave, falling | State change | Continuous rising trend or |
| wave | | falling trend |

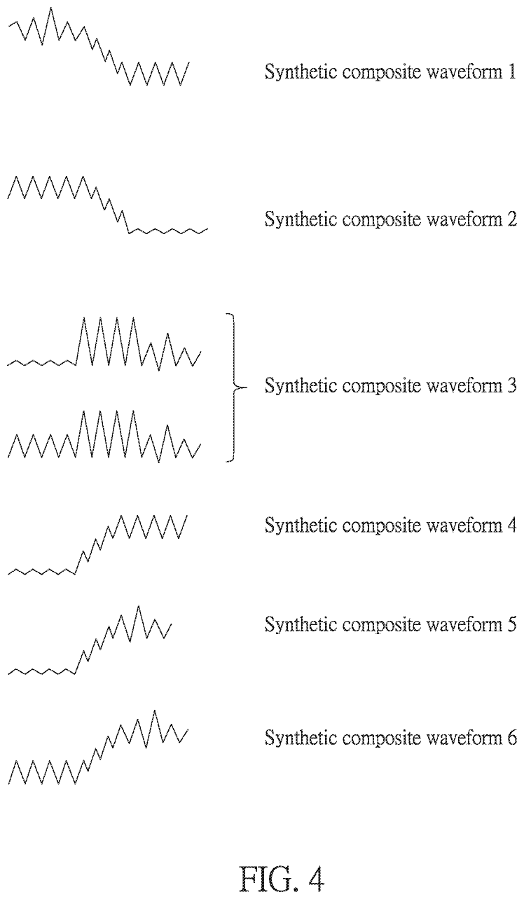

The aforementioned synthetic basic waveforms are further combined to obtain several composite synthetic waveforms as shown in FIG. 4. Each composite synthetic waveform in FIG. 4 corresponds to a physiological state. Taking the sleep state as an example, the correspondences between synthetic composite waveforms and sleep states are shown in Table 2.

TABLE 2
Correspondences between synthetic composite waveforms
and sleep states and identification results

| Composition of waves | Sleep state | Identification result |
|---|---|---|
| Basic independent wave rising | State change | State change |
| and falling waves | | |
| Irregular wave → falling wave | Waking to light | Stages 1 and 2 are waking, |
| → stable medium wave | sleep | and stage 3 is light sleep. |
| Stable medium wave → | Light to deep | Stages 1 and 2 are light sleep, |
| falling wave → stable wavelet | sleep | and stage 3 is deep sleep. |
| Stable wavelet → stable big | SAHS | The first stage is deep sleep, |
| wave → irregular wave | | the second stage is SAHS, and |
| | | the third stage is light sleep. |
| Stable medium wave → stable | SAHS | The first stage is light sleep, |
| big wave → irregular wave | | the second stage is SAHS, and |
| | | the third stage is waking. |
| Stable wavelet → rising wave | Deep to light | The first stage is deep sleep, |
| → stable medium wave | sleep | and the second and third |
| | | stages are light sleep. |
| Stable wavelet → rising wave | Deep sleep to | The first stage is deep sleep, |
| → irregular wave | dreaming | and the second and third |
| | | stages are dreaming. |
| Stable medium wave → rising | Light sleep to | Stages 1 and 2 are light sleep, |
| wave → irregular wave | waking | and stage 3 is waking. |

Taking the non-sleep state (when active) as an example, the correspondences between synthetic basic waveforms and activity states are shown in Table 3:

TABLE 3
Correspondences between synthetic basic waveforms
and activity states and identification features

| Composition of waves | Sleep state | Identification feature |
|---|---|---|
| Stable wavelet | Stable activity state | Fluctuation amplitude ≤ 3 |
| Stable medium wave | Early activity state | 7 ≥ fluctuation amplitude > 3 |
| Stable big wave | Activity changing | Fluctuation amplitude > 7 |
| Irregular wave | Irregular activity | Nonuniform fluctuation |
| | | amplitudes |
| Rising wave, falling | State changing (beginning | Continuous rising or falling |
| wave | and end of activity) | trend |

The aforementioned synthetic basic waveforms are further combined to obtain several synthetic composite waveforms as shown in FIG. 4. Each synthetic composite waveform in FIG. 4 corresponds to a respective physiological state. Taking the non-sleeping phase (active phase) as an example, the correspondences between synthetic composite waveforms and physiological state are shown in Table 4.

TABLE 4
Correspondences between synthetic composite waveforms
and physiological state and identification results

| Composition of waves | Activity state | Identification results |
|---|---|---|
| Basic independent wave | State changing | Beginning and end of |
| rising and falling waves | | activities |
| Irregular wave → falling | Irregular activities turning | Getting up, standing, |
| wave → stable medium | to regular activities | and walking |
| wave | | |
| Stable medium wave → | Early stage of activity | Continuous walking or |
| falling wave → stable | turning to a stable activity | running |
| wavelet | stage | |
| Stable wavelet → stable big | Stable activity stage | Continuous walking |
| wave → irregular wave | turning to different | turning to brisk |
| | activities | walking or running |
| Stable medium wave → | Early stage of activity | After walking a |
| stable big wave → irregular | turning to different | distance, turning to |
| wave | activities | brisk walking or |
| | | running |
| Stable wavelet → rising | Stable activity stage | Continuous walking |
| wave → stable medium | turning to more intense | turning to smooth brisk |
| wave | activities (continuously) | walking or running |
| Stable medium wave | Stable activity stage | Continuous walking |
| rising wave → irregular | turning to more intense | turning to brisk |
| wave | activities, then stopping or | walking or running, |
| | starting | then stopping |
| Stable medium wave → | Early stage of activity | Walking a distance |
| rising wave → irregular | turning to more intense | then turning to brisk |
| wave | activities, then stopping or | walking or running, |
| | starting | then stopping |

### Embodiment 2: Identification of Individual Physiological Status

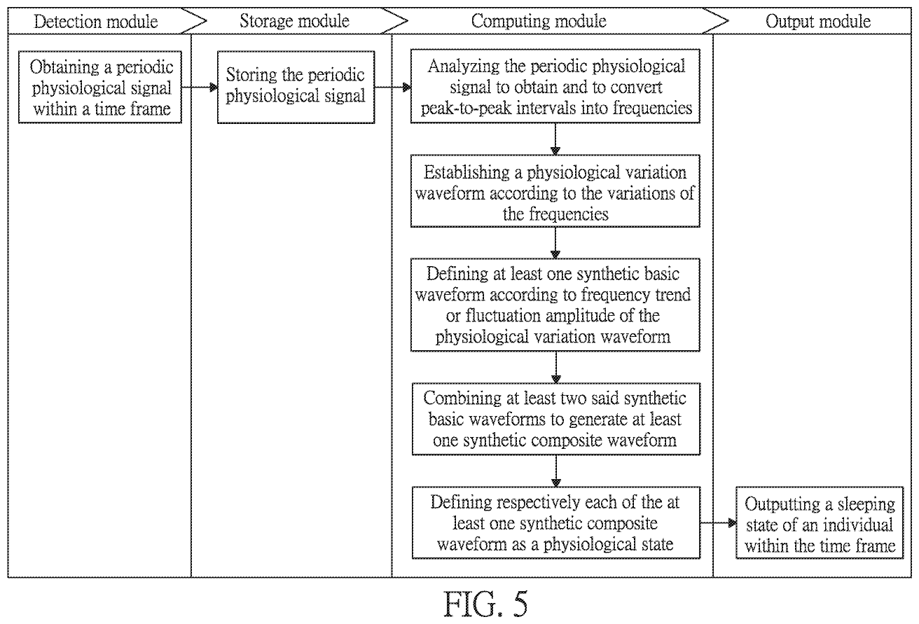

FIG. 5 shows a physiological state analysis system according to an embodiment of the present invention. In order to identify the sleep state of the individual within the examination time, the collected ECG signal or PPG signal is analyzed by the aforementioned method to analyze the frequency variation and to graph the frequency variation per second to obtain a variation waveform. The variation waveform takes one minute as a cycle period, and the synthetic basic waveform with the longest number of seconds staying in a memory in each minute is determined as the presenting synthetic basic waveform of the individual in that minute; and all the presenting synthetic basic waveforms of the individual within the examination time are used for analysis through each of the aforementioned synthetic composite waveforms. And the physiological state of the individual is determined based on the physiological state obtained through the aforementioned correspondences between the synthetic composite waveforms and the physiological state.

Table 5 shows the results of analyzing the sleep state of individual case A through using the synthetic waveforms generated by the ECG signal.

TABLE 5
Analysis results of sleep states and deep
sleep analysis for individual case A

| 1. | Sleep state | | minute | hour |
|---|---|---|---|---|
| | Deep sleep | | 192 | 3.2 |
| | Light sleep | | 208 | 3.5 |
| | Dreaming | | 84 | 1.4 |
| | Waking | | 446 | 7.4 |
| | Invalid | | 2 | 0 |
| 2. | Deep Sleep Analysis | Stages | minute | hour |
| | Deep sleep | | 192 | 3.2 |
| | Total effective deep sleep | 6 | 92 | 1.5 |
| | Longest duration | | 39 | 0.7 |
| | | | minute | # of times |

| | | |
|---|---|---|
| Effective deep sleep | 5 | 1 |
| time interval | 6 | 1 |

| | |
|---|---|
| 7 | 1 |
| 13 | 1 |
| 22 | 1 |
| 39 | 1 |

Based on the results of the above analysis, the sleep quality of the individual can be known, and it can be determined whether the individual sufficiently rests during the sleep.

In addition, according to the aforementioned method, the sleep states whenever SAHS occurs and the number of occurrences of SAHS within the examination time of the individual case A can also be obtained. The analysis results of an individual case A are shown in the table below:

TABLE 6
Analysis results of the sleep state whenever SAHS occurs and
the number of occurrences of SAHS in individual case A

| | | | |
|---|---|---|---|
| 1. | Sleep state when SAHS occurs | SAHS | minute |
| | | SAHS ++ light sleep | 55 |
| | | SAHS ++ dreaming | 34 |
| | | SAHS ++ deep sleep | 19 |
| | | SAHS + light sleep | 54 |
| | | SAHS + dreaming | 27 |
| | | SAHS + deep sleep | 9 |
| 2. | Number of times | SAHS | # of times |
| | | SAHS++ | 102 |
| | | SAHS+ | 205 |
| 3. | AHI | | times/hour |
| | | | 21.3 |

The above analysis can obtain the number of times that the individual has for different degrees of SAHS (the more+signs, the more serious the degree of SAHS) during this time interval of sleep, the state of sleep that the individual was in when SAHS occurred, and the duration of SAHS.

Furthermore, if the individual's heart rhythm is examined during the examination of the individual by the aforementioned method, it can be combined and presented in combination with the time of occurrence, duration, recovery time and heart rate change of the individual when SAHS occurs, as shown in the table below.

TABLE 7
Individual A's SAHS time of occurrence, duration, recovery time, and
corresponding results of heart rate variations during each time interval

| | Time of | | Recovery | Heart rate | Turning | Heart | |
|---|---|---|---|---|---|---|---|
| | occurrence | Duration | time | during | point | rate at | |
| Minute | (minute) | (second) | (second) | occurrence | heart rate | recovery | SAHS |
| 460 | 459.8 | 10.82 | 16.32 | 64.8 | 58.2 | 67.16 | SAHS+ |
| 463 | 462.6 | 10.90 | 11.44 | 58.5 | 65.7 | 59.09 | SAHS+ |
| 478 | 477.4 | 16.05 | 11.68 | 55.1 | 63.5 | 56.48 | SAHS+ |
| 481 | 480.1 | 16.09 | 11.74 | 67.7 | 59.0 | 66.05 | SAHS+ |
| 481 | 480.3 | 10.88 | 11.41 | 59.0 | 66.0 | 58.97 | SAHS+ |
| 491 | 490.1 | 31.69 | 12.70 | 53.9 | 67.4 | 57.18 | SAHS++ |
| 493 | 492.7 | 31.88 | 13.01 | 53.1 | 68.1 | 60.40 | SAHS+ |
| 495 | 494.4 | 21.27 | 17.03 | 52.9 | 63.0 | 53.41 | SAHS+ |
| 497 | 496.8 | 37.12 | 13.39 | 52.8 | 69.7 | 55.66 | SAHS++ |
| 498 | 497.7 | 20.86 | 31.37 | 53.8 | 60.7 | 53.75 | SAHS+ |
| 501 | 500.1 | 11.48 | 22.36 | 57.6 | 69.4 | 55.03 | SAHS++ |
| 508 | 507.1 | 15.80 | 11.29 | 58.5 | 64.9 | 57.68 | SAHS+ |
| 510 | 509.9 | 21.00 | 21.61 | 57.5 | 65.5 | 56.30 | SAHS+ |

Table 8 shows the results of analyzing the sleep states of the synthetic waveforms generated by the individual case B using the PPG signal.

TABLE 8
Analysis results of sleep states and
deep sleep analysis for Individual B

| 1. | Sleep state | | minute | hour |
|---|---|---|---|---|
| | Deep Sleep | | 174 | 2.9 |
| | Light Sleep | | 202 | 3.4 |
| | Dreaming | | 101 | 1.7 |
| | Waking | | 12 | 0.2 |
| | Invalid | | 1 | 0.0 |
| 2. | Deep Sleep Analysis | Stage | minute | hour |
| | Deep Sleep | | 174 | 2.9 |
| | Total effective deep sleep | 8 | 55 | 0.9 |
| | Longest duration | | 12 | 0.2 |
| | | | minute | # of times |

| | | |
|---|---|---|
| Effective deep sleep | 5 | 4 |
| time interval | 6 | 1 |

| | |
|---|---|
| 7 | 1 |
| 10 | 1 |
| 12 | 1 |
| 5 | 4 |

Based on the above analysis results, the sleep quality of the individual can be known, and it can be determined whether the individual has sufficiently rested during the sleep.

In addition, according to the aforementioned method, the sleep states and the number of occurrences of the individual when the SAHS occurred during the examining time can also be detected. The analysis results of the individual case B are shown in the following table:

TABLE 9
Analysis results of sleep states and frequency
of occurrence of SAHS in individual B

| Sleep states when SAHS occurs | SAHS | minute |
|---|---|---|
| | SAHS ++ light sleep | 28 |
| | SAHS ++ dreaming | 3 |
| | SAHS ++ deep sleep | 3 |
| | SAHS + light sleep | 26 |
| | SAHS + dreaming | 5 |
| | SAHS + deep sleep | 5 |
| Number of times | SAHS | # of times |
| | SAHS++ | 35 |
| | SAHS+ | 65 |
| 3. AHI | | times/hour |
| | | 9.3 |

It can be known from the above analysis regarding the number of times that a person had various degrees of SAHS (the more+signs indicating the more serious) during this time interval of sleep, and the state of sleep that the individual was in when SAHS occurred, and the duration of SAHS.

Furthermore, if the individual's heart rhythm is a so detected during the examination of the individual by the aforementioned method, it can be thereby combined and presented in combination with the time of occurrence, duration, recovery time and heart rate change of the individual, as shown in the table below:

TABLE 10
Individual B's SAHS time of occurrence, duration, recovery time, and
corresponding results of heart rate changes during each time interval

| | Time of | | Recovery | Heart rate | Turning | Heart | |
|---|---|---|---|---|---|---|---|
| | occurrence | Duration | time | during | point | rate at | |
| Minute | (minute) | (second) | (second) | occurrence | heart rate | recovery | SAHS |
| 40 | 39.4 | 16.30 | 22.08 | 66.82 | 77.22 | 66.07 | SAHS+ |
| 51 | 50.7 | 31.28 | 12.05 | 62.33 | 72.60 | 61.39 | SAHS+ |
| 52 | 51.2 | 11.40 | 17.24 | 72.60 | 61.39 | 69.80 | SAHS+ |
| 56 | 55.2 | 16.28 | 12.05 | 75.11 | 85.37 | 69.11 | SAHS++ |
| 65 | 64.5 | 27.62 | 19.19 | 85.93 | 64.98 | 76.06 | SAHS+ |
| 119 | 118.6 | 28.42 | 15.48 | 68.32 | 95.71 | 65.60 | SAHS+ |
| 126 | 125.7 | 15.91 | 16.46 | 66.34 | 73.65 | 64.79 | SAHS++ |
| 132 | 131.2 | 17.65 | 14.24 | 64.91 | 86.09 | 61.28 | SAHS+ |
| 132 | 131.4 | 13.10 | 24.96 | 86.09 | 61.28 | 70.44 | SAHS++ |
| 138 | 137.2 | 11.93 | 13.09 | 67.37 | 82.83 | 63.92 | SAHS+ |
| 139 | 138.7 | 37.79 | 14.47 | 64.68 | 87.03 | 64.65 | SAHS+ |

A person of ordinary skill in the art may understand that the implementation of all or part of the processes in the methods of the aforementioned embodiments may be performed by a computer program instructing related hardware. The synthetic basic waveform and/or synthetic composite waveforms may be stored in a computer-readable storage medium. When the computer program is executed, the computer program may include the processes of the foregoing methods. The storage medium may be a magnetic disk, an optical disk, a read-only memory (ROM), a random access memory (RAM), or a cloud storage device.

The aforementioned are preferred embodiments of the present invention. It should be noted that for those of ordinary skill in the art, without departing from the principles of the present invention, certain improvements and retouches of the present invention can still be made which are nevertheless considered as within the protection scope of the present invention.

Even though numerous characteristics and advantages of the present invention have been set forth in the foregoing description, together with details of the structure and function of the invention, the disclosure is illustrative only. Changes may be made in detail, especially in matters of shape, size, and arrangement of parts within the principles of the invention to the full extent indicated by the broad general meaning of the terms in which the appended claims are expressed.

What is claimed is:

1. A method for generating synthetic composite waveforms for determining an individual physiological state, comprising steps of:
(A) obtaining a periodic physiological signal of an individual during a time frame;
(B) analyzing the periodic physiological signal to obtain and to convert peak-to-peak intervals of the periodic physiological signal into frequencies;
(C) establishing a physiological variation waveform according to the variations of the frequencies;
(D) defining at least one synthetic basic waveform according to frequency trend or fluctuation amplitude of the physiological variation waveform;
(E) combining at least two said synthetic basic waveforms to generate at least one synthetic composite waveform; and
(F) defining respectively each of the at least one synthetic composite waveform as a physiological state.

2. The method for generating synthetic composite waveforms as claimed in claim 1, wherein the periodic physiological signal is a single signal.

3. The method for generating synthetic composite waveforms as claimed in claim 1, wherein the synthetic basic waveform includes a stable waveform, a state waveform, and an irregular waveform, wherein the stable waveform means that the amplitude of the fluctuation is stable, the state waveform means that the amplitude of the fluctuation rises gradually or falls gradually, and the irregular waveform means that the amplitude of the fluctuation exhibits irregular variations.

4. The method for generating synthetic composite waveforms as claimed in claim 1, wherein the physiological state is a sleeping state or a non-sleeping state.

5. The method for generating synthetic composite waveforms as claimed in claim 1, further comprising a step of:
(G) determining the physiological state of the individual during the time frame.

6. A synthetic composite waveform generation system for determining an individual's physiological state, including:
A. a detection module, for obtaining a periodic physiological signal of an individual within a time frame;
B. a storage module, for storing the periodic physiological signal; and
C. a computing module, for
c1. analyzing the periodic physiological signal to obtain peak-to-peak intervals of the periodic physiological signal, and converting the peak-to-peak intervals into frequencies;
c2. establishing a physiological variation waveform according to variations in the frequencies;
c3. defining at least one synthetic basic waveform according to the frequency trend or fluctuation amplitude of the physiological variation waveform;
c4. combining at least two said synthetic basic waveforms to generate at least one synthetic composite waveform; and
c5. defining respectively each of the at least one synthetic composite waveform as a physiological state.

7. The synthetic composite waveform generation system as claimed in claim 6, wherein the physiological signal is a single signal.

8. The synthetic composite waveform generation system as claimed in claim 6, wherein the synthetic basic waveform includes a stable waveform, a state waveform, and an irregular waveform, wherein the stable waveform means that the amplitude of the fluctuation is stable, the state waveform means that the amplitude of the fluctuation rises gradually or falls gradually, and the irregular waveform means that the amplitude of the fluctuation exhibits irregular variations.

9. The synthetic composite waveform generation system as claimed in claim 6, wherein the physiological state is a sleeping state or a non-sleeping state.

10. The synthetic composite waveform generation system as claimed in claim 6, further comprising an output module for outputting the physiological state of the individual during the time frame.
